(19) United States
(12) Patent Application Publication
(10) Pub. No.: US 2022/0383064 A1
(43) Pub. Date: Dec. 1, 2022

(54) INFORMATION PROCESSING METHOD AND DEVICE

(71) Applicant: Baidu.com Times Technology (Beijing) Co., Ltd., Beijing (CN)

(72) Inventors: Shuangli LI, Beijing (CN); Jingbo ZHOU, Beijing (CN); Tong XU, Beijing (CN); Liang HUANG, Beijing (CN); Fan WANG, Beijing (CN); Haoyi XIONG, Beijing (CN); Weili HUANG, Beijing (CN); Hui XIONG, Beijing (CN); Dejing DOU, Beijing (CN)

(73) Assignee: Baidu.com Times Technology (Beijing) Co., Ltd., Beijing (CN)

(21) Appl. No.: 17/565,910

(22) Filed: Dec. 30, 2021

(30) Foreign Application Priority Data
May 18, 2021 (CN) 202110543978.2

(51) Int. Cl. G06N 3/04

(52) U.S. Cl. CPC G06N 3/04

(57) ABSTRACT

The present disclosure discloses an information processing method and device, and relates to the field of artificial intelligence, in particular, to a graph neural network in the field of deep learning. A specific implementation solution according to an embodiment includes determining initial representation of edges connected between a plurality of atoms in a molecule based on three-dimensional structure information of the molecule; determining first representation of a neighbor edge of each of the atoms based on the initial representation of the edges, the neighbor edge of each of the atoms indicating at least one edge connected with each of the atoms; determining first representation of each of the atoms based on the first representation of the neighbor edge of each of the atoms; determining feature representation for characterizing the molecule based on the first representation of each of the atoms.

### CROSS-REFERENCE TO RELATED APPLICATION

The present disclosure claims priority to Chinese Patent Application Priority No. 202110543978.2, filed with the Chinese Patent Office on May 18, 2021, and titled “Information processing method and device”, which disclosure is incorporated herein in its entirety by reference

### TECHNICAL FIELD

The present disclosure relates to the field of artificial intelligence, and in particular, to a graph neural network in the field of deep learning. More specifically, the present disclosure relates to an information processing method and device, an electronic device, a computer readable storage medium, and a computer program product.

### BACKGROUND

In the fields of computational biology and computational chemistry, effective molecular characterization is essential for the understanding and accurate prediction of various biochemical properties. A network structure diagram composed of multiple types of atomic interactions can be used to characterize molecules.

### SUMMARY

The present disclosure provides an information processing method and device, a device and a storage medium.

According to a first aspect of the present disclosure, an information processing method is provided. The method includes initial representation of edges connected between multiple atoms in a molecule is determined based on three-dimensional structure information of the molecule. The method further includes first representation of a neighbor edge of each of the atoms is determined based on the initial representation of the edges, the neighbor edge of each of the atoms indicating at least one edge connected with each of the atoms. The method further includes first representation of each of the atoms is determined based on the first representation of the neighbor edge of each of the atoms. The method further includes feature representation for characterizing the molecule is determined based on the first representation of each of the atoms.

According to a second aspect of the present disclosure, an information processing device is provided. The device includes an initial representation determination module. The initial representation determination module is configured to determine initial representation of edges connected between multiple atoms in a molecule based on three-dimensional structure information of the molecule. The device further includes an edge determination module. The edge determination module is configured to determine first representation of a neighbor edge of each of the atoms based on the initial representation of the edges. The neighbor edge of each of the atoms indicates at least one edge connected with each of the atoms. The device further includes an atom determination module. The atom determination module is configured to determine first representation of each of the atoms based on the first representation of the neighbor edge of each of the atoms. The device further includes a characterization module. The characterization module is configured to determine feature representation for characterizing the molecule based on the first representation of each of the atoms.

According to a third aspect of the present disclosure, an electronic device is provided. The electronic device includes at least one processor and a memorizer in communication connection with the at least one processor. The memorizer stores an instruction capable of being performed by the at least one processor. The instruction is performed by the at least one processor, to cause the at least one processor to perform the method according to the first aspect of the present disclosure.

According to a fourth aspect of the present disclosure, a non-transitory storage medium storing a computer instruction is provided. The computer instruction is used for a computer to perform the method according to the first aspect of the present disclosure.

According to a fifth aspect of the present disclosure, a computer program product is provided. The method according to the first aspect of the present disclosure is implemented when the computer program is performed by a processor.

It is to be understood that, the content described in this section is not intended to identify the key or important features of the embodiments of the present disclosure, nor is it intended to limit the scope of the present disclosure. Other features of the present disclosure will become easy to understand through the following description.

### BRIEF DESCRIPTION OF THE DRAWINGS

Drawings are used to better understand the solution, and are not intended to limit the present disclosure, wherein

FIG. 1 is an architecture diagram of a system for characterizing molecules according to an embodiment of the present disclosure.

FIG. 2 is a schematic diagram of a combination process for edges according to an embodiment of the present disclosure.

FIG. 3 is a schematic diagram of a combination process for atoms according to an embodiment of the present disclosure.

FIG. 4 is a flowchart of a method for characterizing molecules according to an embodiment of the present disclosure.

FIG. 5 is a schematic block diagram of a device for characterizing molecules according to an embodiment of the present disclosure.

FIG. 6 is a block diagram of an electronic device configured to implement a method for molecule characterization according to an embodiment of the present disclosure.

### DETAILED DESCRIPTION OF THE INVENTION

Exemplary embodiments of the present disclosure are described in detail below with reference to the drawings, including various details of the embodiments of the present disclosure to facilitate understanding, and should be regarded as merely exemplary. Thus, those of ordinary skilled in the art shall understand that, variations and modifications can be made on the embodiments described herein, without departing from the scope and spirit of the present disclosure. Likewise, for clarity and conciseness, descriptions of well-known functions and structures are omitted in the following description.

As described above, in the fields of computational biology and computational chemistry, effective molecular characterization is essential for the understanding and accurate prediction of various biochemical properties. A molecule is essentially a network structure diagram composed of multiple types of atomic interactions. In addition to topological structure information, the network structure diagram of the molecule also includes key spatial structure information, for example, an angle and distance between atoms forming the molecule. At present, there is still a need for a method that can better characterize the three-dimensional structure information of the molecule.

According to an embodiment of the present disclosure, a solution for characterizing molecules is provided. In the solution, initial representation of edges connected between multiple atoms in a molecule is determined based on three-dimensional structure information of a molecule. The solution further includes first representation of a neighbor edge of each of the atoms is determined based on the initial representation of the edges. The neighbor edge of each of the atoms indicates at least one edge connected with each of the atoms. The solution further includes first representation of each of the atoms is determined based on the first representation of the neighbor edge of each of the atoms. The solution further includes feature representation for characterizing the molecule is determined based on the first representation of each of the atoms. In this way, the representation of the atoms and the edges can be interactively generated to integrate the information of the neighbor atoms so as to better characterize the molecule.

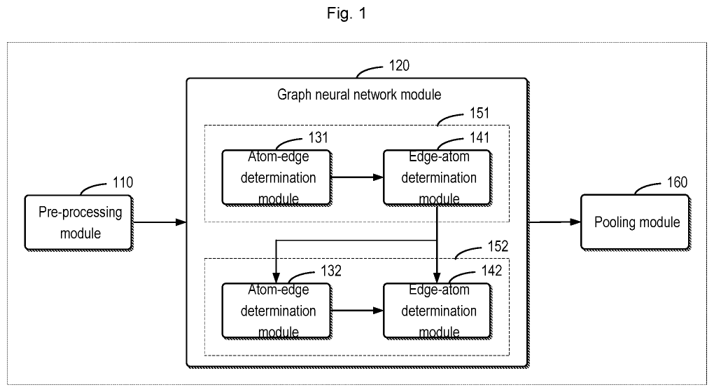

FIG. 1 is an architecture diagram of a system 100 for characterizing molecules according to an embodiment of the present disclosure. As shown in the figure, the system 100 may include a pre-processing module 110, a graph neural network module 120, and a pooling module 160. The graph neural network module 120 may include atom-edge determination modules 131 and 132 (hereinafter collectively referred to as 130) and edge-atom determination modules 141 and 142 (hereinafter collectively referred to as 140). The graph neural network module may include multiple layers L, for example, a first layer 151 and a second layer 152. In the first layer 151, the atom-edge determination module 131 and the edge-atom determination module 141 may be performed. In the second layer 152, the atom-edge determination module 132 and the edge-atom determination module 142 may be performed. It is to be understood that, the system 100 shown in FIG. 1 is merely exemplary, and should not constitute any limitation to the functions and scope of the implementation described in the present disclosure. For example, the graph neural network module 120 may include more than two atom-edge determination modules and edge-atom determination modules.

In some embodiments, the pre-processing module 110 may receive the three-dimensional structure information 101 of the molecule. The three-dimensional structure information 101 of the molecule may include types and space distribution of the atoms forming the molecule. Additionally, the three-dimensional structure information 101 of the molecule may further include the type, physical and chemical properties, name, and the like of the molecule. The three-dimensional structure information 101 of the molecule may be in a form of a molecular diagram, or in a form that may represent the three-dimensional structure information 101 of the molecule. The pre-processing module 110 determines the initial representation of the atoms in the molecule based on the three-dimensional structure information 101 of the molecule. The initial representation of the atoms may be initial vector representation generated based on the properties of the atoms, space distribution, the properties of the molecule and other information. The initial representation of the atoms may be determined by utilizing multiple methods, which is not limited by the scope of the present disclosure.

The pre-processing module 110 may also determine the space distribution of the atoms based on the three-dimensional structure information 101 of the molecule. The pre-processing module 110 may construct an edge between the atoms of which distance between each other is less than a threshold distance (for example, 3 angstroms). The pre-processing module 110 may determine the characterization of the distance between the atoms connected by the edge based on the three-dimensional structure information 101 of the molecule. In some embodiments, the characterization of the distance may be obtained by vectorizing the distance between the atoms. For example, the distance between the atoms may be discretized to obtain the one-hot encoding of the distance. Based on the one-hot encoding of the distance, the characterization of the distance may be obtained.

The pre-processing module 110 may also determine an included angle between neighbor edges connected with the same atom based on the three-dimensional structure information 101 of the molecule. In some embodiments, a polar coordinate system may be utilized to represent the three-dimensional structure information 101 of the molecule. In this case, the included angle between the neighbor edges may be calculated more easily. For example, a first edge connected with a first atom may be taken as a polar axis, and the first atom is taken as a pole. The pre-processing module 110 may determine an included angle between each of the other edges except the first edge in the neighbor edge connected with the first atom and the first edge to obtain multiple included angles. In some embodiments, the included angle may be represented by using (θ, φ), and θ and φ may be in a range of 0 to 180°.

The pre-processing module 110 may input the initial representation of the atoms, the characterization of the distance between the atoms and the multiple included angles determined based on the three-dimensional structure information 101 of the molecule into the graph neural network module 120. The graph neural network module 120 may be a graph neural network that outputs the feature representation of the molecule based on the above input data.

In some embodiments, the atom-edge determination module 131 may determine the initial representation of the edges connecting the atoms based on the initial representation of the atoms and the characterization of the distance between the atoms. The initial representation of the edges may be one-dimensional vector representation. In some embodiments, the initial representation of the atoms connected by the edge and the characterization of the distance may be concatenated to determine the initial representation of the edges. Alternatively, the average value of the initial representation of the connected atoms and the characterization of the distance may be concatenated to determine the initial representation of the edges.

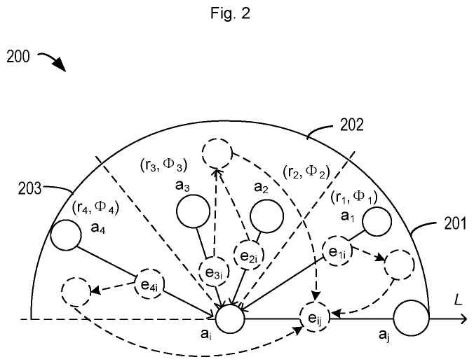

The atom-edge determination module 131 also determines the first representation of the neighbor edge based on the initial representation of the neighbor edge. A combination process for the edges will be described in detail below with reference to FIG. 2. FIG. 2 is a schematic diagram of a combination process 200 for edges according to an embodiment of the present disclosure. FIG. 2 illustrates the first atom aᵢ and neighbor edges eᵢⱼ, e₁ᵢ, e₂ᵢ, e₃ᵢ, and e₄ᵢ connected with the first atom. It is to be understood that, aᵢ may also be used to represent the characterization of the first atom. Similarly, eᵢⱼ, e₁ᵢ, e₂ᵢ, e₃ᵢ, and e₄ᵢ may also be used to represent the characterization of the neighbor edges. The atom-edge determination module 131 determines the first representation of the neighbor edges eᵢⱼ, e₁ᵢ, e₂ᵢ, e₃ᵢ, and e₄ᵢ based on the initial representation of the neighbor edges eᵢⱼ, e₁ᵢ, e₂ᵢ, e₃ᵢ, and e₄ᵢ of the first atom aᵢ. In some embodiments, the atom-edge determination module 131 may select the first edge eᵢⱼ in the neighbor edges, and determines first representation of the first edge eᵢⱼ based on a combination of the initial representation of the other edges eₖᵢ (for example, eᵢⱼ, e₁ᵢ, e₂ᵢ, e₃ᵢ, and e₄ᵢ) except the first edge eᵢⱼ. The atom-edge determination module 131 may combine the initial representation of the other edges eₖᵢ as the first representation of the first edge eᵢⱼ based on the multiple included angles between the neighbor edges determined by the pre-processing module 110.

In some embodiments, the atom-edge determination module 131 may divide the other edges eₖᵢ into different angle domains based on the multiple included angles between the neighbor edges. For example, a formula (1) may be utilized to calculate indexes Indₖᵢ of the angle domains at which the other edges eₖᵢ are located.

$${Ind}_{ki} = {D}_{A} ( {e}_{ki} , {e}_{ij} , N ) = ⌈ N \cdot \frac{{ϕ}_{kij}}{180} ⌉ \qquad ( 1 )$$

Wherein, D_{A} represents an angle domain divider, ┌·┐ represents a rounding symbol, ϕₖᵢⱼ ∈ [0, 180°] represents an included angle between the edges eₖᵢ and eᵢⱼ, and N represents the quantity of the angle domains. As shown in FIG. 2, the other edges e₁ᵢ, e₂ᵢ and e₃ᵢ, and e₄ᵢ are respectively divided into the angle domains 201, 202 and 203. It is to be understood that, angle domain division shown in FIG. 2 is merely exemplary. In some embodiments, the angle domains may be divided based on the value of the multiple included angles θ and φ. It is to be understood that, the repeated combination of the same edge may be reduced by limiting ϕₖᵢⱼ ∈ [0, 180°]. ϕₖᵢⱼ ∈ [0, 360°] and other rules may be set for a combination process for the neighbor edges.

In some embodiments, the atom-edge determination module 131 may determine the attention weight of the other edges eₖᵢ to the first edge eᵢⱼ in each of the angle domains. For example, the attention weight of the other edge e₁ᵢ to the first edge eᵢⱼ in the angle domain 201 may be determined; the attention weight of the other edges e₂ᵢ and e₃ᵢ to the first edge eᵢⱼ in the angle domain 202 may be determined; and the attention weight of the other edge eₖᵢ to the first edge eᵢⱼ in the angle domain 203 may be determined. Formulas (2) to (3) may be utilized to calculate the attention weight of the other edges eₖᵢ to the first edge eᵢⱼ in the angle domain q.

$${attn}_{q}^{l} ( {e}_{ij} , {e}_{ki} ) = {u}_{l , q}^{T} \cdot tanh ( {W}_{e , q}^{( l )} \cdot [ {e}_{ij}^{( l )} ‖ {e}_{ki}^{( l )} ] + {b}_{e , q}^{( l )} ) \qquad ( 2 )$$

$${α}_{ki , q}^{( l )} = \frac{exp ( {attn}_{q}^{l} ( {e}_{ij} , {e}_{ki} ) )}{{\sum }_{{e}_{ti} ∈ {N}_{c}^{q} ( {e}_{ij} )} exp ( {attn}_{q}^{l} ( {e}_{ij} , {e}_{ti} ) )} \qquad ( 3 )$$

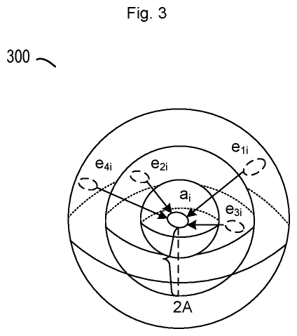

A function attn_{q}^{l} may calculate an importance factor of the neighbor edge eₖᵢ to eᵢⱼ in a layer 1. In the calculation of the atom-edge determination module 131, the layer 1 is the first layer 151. As shown in the formula (2), the importance factor may be calculated by means of concatenating of eₖᵢ and eᵢⱼ. w_{e,q}^{(l)}, b_{e,q}^{(l)}, and u_{l,q}^{T} are trainable parameter matrices. α_{ki,q}^{(l)} represents the attention weight of the neighbor edges eₖᵢ in the specific angle domain q. As shown in FIG. 3, a softmax function may be utilized to standardize the importance factor to obtain α_{ki,q}^{(l)}.

In some embodiments, based on the attention weight α_{ki,q}^{(l)} of the other edges eₖᵢ to the first edge eᵢⱼ in each of the angle domains q, the atom-edge determination module 131 may determine weighted initial representation for each of the angle domains q through weighted summation of the initial representation of the other edges in each of the angle domains q. For example, a formula (4) may be utilized to calculate the weighted initial representation m_{ij,q}^{(l)} for the angle domain q.

$${m}_{ij , q}^{( l )} = {\sum }_{{e}_{ki} ∈ {N}_{c}^{q} ( {e}_{ij} )} {α}_{ki , q}^{( l )} \cdot {e}_{ki}^{( l )} , 1 \leq q \leq N \qquad ( 4 )$$

The atom-edge determination module 131 may also determine the characterization of the combined first edge eᵢⱼ, that is, the first representation of the first edge eᵢⱼ, by concatenating the weighted initial representation m_{ij,q}^{(l)} for each of the angle domains q. For example, a formula (5) may be utilized to calculate the first representation of the first edge eᵢⱼ through concatenating.

eᵢⱼ^{(l)}=[m_{ij,1}^{(l)}∥m_{ij,2}^{(l)}∥ . . . ∥m_{ij,N}^{(l)}] (5)

Similarly, the atom-edge determination module 131 may determine first representation of all edges in the molecule. In this way, the information of the neighbor edges of each of the atoms may be combined with the information of the edge connected with the atom, so that the first representation of the edge may better characterize the edge and a surrounding molecular structure, thereby better characterizing the molecule.

Further referring to FIG. 1, the atom-edge determination module 131 may input the determined first representation of the neighbor edges into the edge-atom determination module 141. The edge-atom determination module 141 determines the first representation of the first atom based on the first representation of the neighbor edges. A combination process for the atoms will be described in detail below with reference to FIG. 3. FIG. 3 is a schematic diagram of a combination process 300 for atoms according to an embodiment of the present disclosure. FIG. 3 illustrates the first atom aᵢ and the neighbor edges eᵢⱼ, e₁ᵢ, e₂ᵢ, e₃ᵢ, and e₄ᵢ (which are recorded as the neighbor edge eₖᵢ below) of the first atom aᵢ. It is to be understood that, the neighbor edges eᵢⱼ, e₁ᵢ, e₂ᵢ, e₃ᵢ, and e₄ᵢ are merely exemplary.

In some embodiments, the edge-atom determination module 141 may determine a distance between the neighbor edges eᵢⱼ, e₁ᵢ, e₂ᵢ, e₃ᵢ, and e₄ᵢ and the first atom aᵢ. The distance between the neighbor edge and the first atom aᵢ may be a distance between a second atom (the atoms a₁, a₂, a₃, and a₄ shown in FIG. 2) connected by the neighbor edge and the first atom aᵢ. The edge-atom determination module 141 may determine the attention weight of the neighbor edge eₖᵢ to the first atom aᵢ based on the distance. For example, a formula (6) to (7) may be utilized to calculate the attention weight of the neighbor edge eₖᵢ to the first atom aᵢ.

wₖᵢ^{(l)}=LeakyRelu(vₗ^{T}·[ēₖᵢ^{(l)}∥āᵢ^{(l)}∥W_{d}^{(l)}dₖᵢ]) (6)

$${β}_{ki}^{( l )} = \frac{exp ( {w}_{ki}^{( l )} )}{{\sum }_{{e}_{ki} ∈ {N}_{c} ( {a}_{i} )} exp ( {w}_{ki}^{( l )} ) )} \qquad ( 7 )$$

A function LeakyRelu may calculate an importance factor of the neighbor edge eₖᵢ to aᵢ in a layer 1. In the calculation of the edge-atom determination module 141, the layer 1 is the first layer 151. As shown in a formula (6), the importance factor wₖᵢ^{(l)} may be calculated by means of the concatenating of ēₖᵢ^{(l)}, āᵢ^{(l)} and W_{d}^{(l)}dₖᵢ. ēₖᵢ^{(l)} and āᵢ^{(l)} are respectively the first representation of the converted neighbor edge eₖᵢ and the initial representation of the converted first atom aᵢ. By converting the first representation of the neighbor edge eₖᵢ and the initial representation of the first atom aᵢ, the first representation of the neighbor edge eₖᵢ and the initial representation of the first atom aᵢ may be converted to a same feature space, so that a follow-up concatenating operation is realized. W_{d}^{(l)} and vₗ^{T} are trainable parameter matrices.

βₖᵢ^{(l)} represents the attention weight of the neighbor edge eₖᵢ to the first atom aᵢ. As shown in a formula (7), the importance factor wₖᵢ^{(l)} may be standardized by using the softmax function to obtain βₖᵢ^{(l)}. Based on the attention weight βₖᵢ^{(l)} of the neighbor edge eₖᵢ to the first atom aᵢ, the edge-atom determination module 141 may determine the first representation of the first atom aᵢ by determining a weighted average of the first representation of the neighbor edge.

Additionally, the edge-atom determination module 141 may calculate the attention weight of the neighbor edge eₖᵢ to the first atom aᵢ for multiple times by using a multi-head attention algorithm. In this case, a formula (8) may be utilized to calculate the weighted average of the first representation of the neighbor edge, so as to determine the first representation of the first atom aᵢ.

$${a}_{i}^{( l )} = \frac{1}{C} {\sum }_{c = 1}^{C} {\sum }_{{e}_{ki} ∈ {N}_{c} ( {a}_{i} )} {β}_{ki , c}^{( l )} \cdot {\tilde{e}}_{ki , c}^{( l )} \qquad ( 8 )$$

Where, C represents the quantity of attention heads.

Similarly, the edge-atom determination module 141 may determine the first representation of all atoms in the molecule. In this way, by combining the information of the neighbor edge of each of the atoms into the first representation of the atom, the first representation of the atom may be better characterize the atom and a surrounding molecular structure.

Further referring to FIG. 1, by means of the atom-edge determination module 131 and the edge-atom determination module 141, angle and distance factors in the space distribution of the atom may be fully considered when the molecule is characterized, so that the molecule is better characterized. In some embodiments, the graph neural network module 120 may also use the atom-edge determination module 132 and the edge-atom determination module 142 to continuously iterate representation of the atoms and the edges in a second layer 152.

Similarly, the atom-edge determination module 132 may determine second representation of the neighbor edge of each of the atoms based on the first representation of each of the atoms. For example, the atom-edge determination module 132 may determine second representation of the edges by concatenating the first representation of the atoms connected by the neighbor edges and the characterization of the distance. The atom-edge determination module 132 may determine third representation of the neighbor edges based on the second representation of the neighbor edges. For example, the information of the neighbor edges may be transmitted into the third representation of a target edge in the neighbor edges based on a combination of angles. The edge-atom determination module 142 may determine second representation of the first atom based on the third representation of the neighbor edges of the first atom. For example, the information of the neighbor edges and neighbor atoms may be transmitted into the second representation of the atoms based on a combination of distances. Additionally, the graph neural network module 120 may also utilize the follow-up iteration in other layers to determine final representation of the atoms and the edges. In this way, the representation of the atoms and the edges can be interactively generated. The space structure information of the atoms is integrated based on the combination of the angles and the distances, so that the molecule is better characterized.

Further referring to FIG. 1, a pooling module 160 may determine the feature representation for characterizing the molecule based on the final representation aᵢ^{(L)} of all atoms in the molecule. The feature representation for characterizing the molecule may be one-dimensional vector representation. In some embodiments, summation pooling .h=Σₐ_{ᵢ} aᵢ^{(L)} may be utilized to calculate the feature representation h of the molecule. In some embodiments, the feature representation of the molecule may be determined by calculating a maximum value of the final representation aₗ^{(L)}| of all atoms. Additionally or alternatively, the feature representation of the molecule may be determined based on the final representation of all atoms aₗ^{(L)} and the final representation of all edges.

In some embodiments, the system 100 or the graph neural network module 120 may be trained based on a downstream task of molecular characterization. Different loss functions may be selected for different downstream tasks, so as to train the system 100 or the graph neural network module 120. For example, the L1 loss function may be selected when molecular property prediction is performed. A cross entropy function may be selected when binary-classification DTI property prediction is performed. Limitations are not imposed in the scope of the present disclosure thereto.

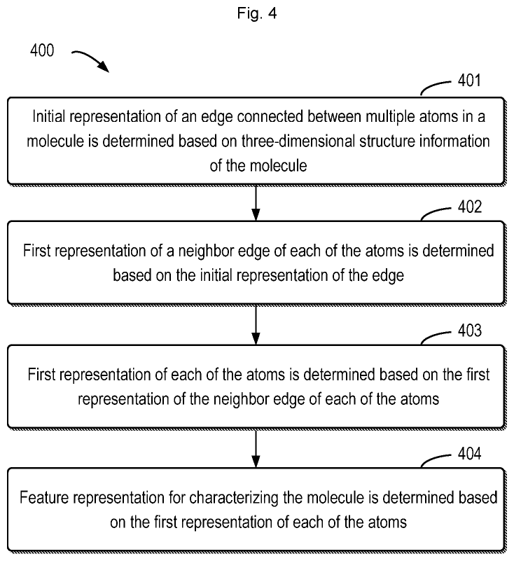

FIG. 4 is a flowchart of an implemented method 400 for characterizing molecules according to the present disclosure. The method 400 may be implemented at the system 100. At 401, initial representation of an edge connected between multiple atoms in a molecule is determined based on three-dimensional structure information of the molecule. At 402, first representation of a neighbor edge of each of the atoms is determined based on the initial representation of the edge. The neighbor edge of each of the atoms indicates at least one edge connected with each of the atoms.

In some embodiments, determining the first representation of the neighbor edge of each of the atoms includes: an included angle between each of the other edges except a first edge in the neighbor edge of a first atom in the multiple atoms and the first edge is determined to obtain multiple included angles; and the first representation of the first edge is determined based on the multiple included angles and initial representation of the other edges.

In some embodiments, determining the first representation of the first edge includes: the other edges are divided into different angle domains based on the multiple included angles; weighted initial representation for each of the angle domains through weighted summation of the initial representation of the other edges in each of the angle domains is determined based on attention weight of the other edges in each of the angle domains to the first edge; and concatenating the weighted initial representation for each of the angle domains as the first representation of the first edge.

At 403, first representation of each of the atoms is determined based on the first representation of the neighbor edge of each of the atoms. In some embodiments, determining the first representation of each of the atoms includes: a distance between the neighbor edge of the first atom in the multiple atoms and the first atom is determined, the distance between the neighbor edge and the first atom indicating a distance between a second atom connected with the neighbor edge and the first atom; the attention weight of the neighbor edge of the first atom to the first atom is determined based on the distance; and an weighted average of the first representation of the neighbor edge of the first atom is determined based on the attention weight as first representation of the first atom.

At 404, feature representation for characterizing the molecule is determined based on the first representation of each of the atoms. In some embodiments, determining the feature representation characterizing the molecule includes: second representation of the edge is determined based on the first representation of each of the atoms; third representation of the neighbor edge of each of the atoms is determined based on the second representation of the edge; second representation of each of the atoms is determined based on the third representation of the neighbor edge of each of the atoms; and the feature representation characterizing the molecule is determined based on the second representation of each of the atoms.

In some embodiments, a polar coordinate system is utilized to represent the three-dimensional structure information of the molecule.

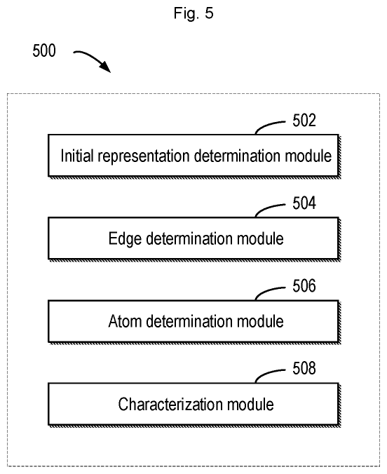

FIG. 5 is a schematic block diagram of a device 500 for characterizing molecules according to an embodiment of the present disclosure. As shown in FIG. 5, the device 500 includes an initial representation determination module 502. The initial representation determination module is configured to determine initial representation of an edge connected between multiple atoms in a molecule based on three-dimensional structure information of the molecule. The device 500 further includes an edge determination module 504. The edge determination module is configured to determine first representation of a neighbor edge of each of the atoms based on the initial representation of the edge. The neighbor edge of each of the atoms indicates at least one edge connected with each of the atoms. The device 500 further includes an atom determination module 506. The atom determination module is configured to determine first representation of each of the atoms based on the first representation of the neighbor edge of each of the atoms. The device 500 further includes a characterization module 508. The characterization module is configured to determine feature representation for characterizing the molecule based on the first representation of each of the atoms. It is to be understood that, the initial representation determination module 502, the edge determination module 504, the atom determination module 506, and the characterization module 508 may implement part or all of functions of the pre-processing module 110, the graph neural network module 120, and the pooling module 160 as shown in FIG. 1.

In some embodiments, the edge determination module 504 includes: an included angle determination sub-module, configured to determine an included angle between each of the other edges except a first edge in the neighbor edge of a first atom in the multiple atoms and the first edge to obtain multiple included angles; and a combination sub-module, configured to determine the first representation of the first edge based on the multiple included angles and initial representation of the other edges.

In some embodiments, the combination sub-module includes: a division sub-module, configured to divide the other edges into different angle domains based on the multiple included angles; a summation sub-module, configured to determine weighted initial representation for each of the angle domains through weighted summation of the initial representation of the other edges in each of the angle domains based on attention weight of the other edges in each of the angle domains to the first edge; and a concatenating sub-module, configured to concatenate the weighted initial representation for each of the angle domains as the first representation of the first edge.

In some embodiments, the atom determination module 506 includes: a distance determination sub-module, configured to determine a distance between the neighbor edge of the first atom in the multiple atoms and the first atom, the distance between the neighbor edge and the first atom indicating a distance between a second atom connected with the neighbor edge and the first atom; a weight determination sub-module, configured to determine the attention weight of the neighbor edge of the first atom to the first atom based on the distance; and a weighted average sub-module, configured to determine an weighted average of the first representation of the neighbor edge of the first atom based on the attention weight as first representation of the first atom.

In some embodiments, the characterization module 508 includes: a second edge determination sub-module, configured to determine second representation of the edges based on the first representation of each of the atoms; a second edge determination sub-module, configured to determine third representation of the neighbor edge of each of the atoms based on the second representation of the edges; a second atom determination sub-module, configured to determine second representation of each of the atoms based on the third representation of the neighbor edge of each of the atoms; and a second characterization sub-module, configured to determine the feature representation for characterizing the molecule based on the second representation of each of the atoms.

In some embodiments, a polar coordinate system is utilized to represent the three-dimensional structure information of the molecule.

According to an embodiment of the present disclosure, the present disclosure further provides an electronic device, a readable storage medium, and a computer program product.

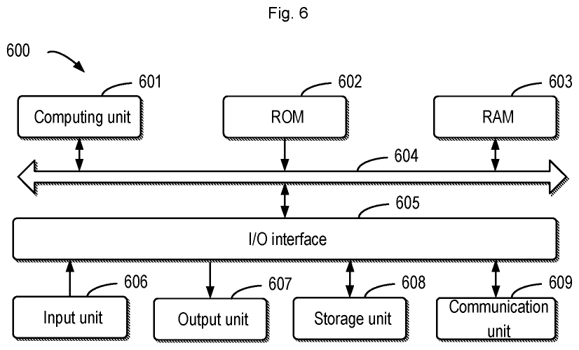

FIG. 6 is a schematic block diagram of an example electronic device 600 configured to implement an embodiment of the present disclosure. The electronic device is intended to represent various forms of digital computers, such as laptop computers, desktop computers, workbenches, personal digital assistants, servers, blade servers, mainframe computers, and other suitable computers. The electronic device may also express various forms of mobile devices, such as personal digital processing, cellular phones, smart phones, wearable devices, and other similar computing devices. The components shown herein, connections and relationships of the components, and functions of the components are merely examples, and are not intended to limit the implementation of the present disclosure described and/or required herein.

As shown in FIG. 6, the device 600 includes a computing unit 601. The computing unit may perform various appropriate actions and processing operations according to a computer program stored in a Read-Only Memory (ROM) 602 or a computer program loaded from a storage unit 608 into a Random Access Memory (RAM) 603. In the RAM 603, various programs and data required for the device 600 may also be stored. The computing unit 601, the ROM 602, and the RAM 603 are connected with each other by using a bus 604. An Input/Output (I/O) interface 605 is also connected with the bus 604.

Multiple components in the device 600 are connected with the I/O interface 605, and include: an input unit 606, such as a keyboard and a mouse; an output unit 607, such as various types of displays and loudspeakers; the storage unit 605 such as a disk and an optical disc; and a communication unit 609, such as a network card, a modem, and a wireless communication transceiver. The communication unit 609 allows the device 600 to exchange information/data with other devices through a computer network, such as the Internet, and/or various telecommunication networks.

The computing unit 601 may be various general and/or special processing assemblies with processing and computing capabilities. Some examples of the computing unit 601 include, but are not limited to, a Central Processing Unit (CPU), a Graphics Processing Unit (GPU), various dedicated Artificial Intelligence (AI) computing chips, various computing units for running machine learning model algorithms, a Digital Signal Processor (DSP), and any appropriate processors, controllers, microcontrollers, etc. The computing unit 601 performs the various methods and processing operations described above, for example, the method 400. For example, in some embodiments, the method 400 may be implemented as a computer software program, which is tangibly included in a machine-readable medium, such as the storage unit 608. In some embodiments, part or all of the computer programs may be loaded and/or installed on the device 600 via the ROM 602 and/or the communication unit 609. When the computer program is loaded into the RAM 603 and performed by the computing unit 601, one or more steps of the method 400 described above may be performed. Alternatively, in other embodiments, the computing unit 601 may be configured to perform the method 400 in any other suitable manners (for example, by means of firmware).

The various implementations of systems and technologies described herein may be implemented in a digital electronic circuit system, an integrated circuit system, a Field Programmable Gate Array (FPGA), an Application-Specific Integrated Circuit (ASIC), an Application-Specific Standard Product (ASSP), a System-On-Chip (SOC), a Complex Programmable Logic Device (CPLD), computer hardware, firmware, software, and/or a combination thereof. These various implementations may include: being implemented in one or more computer programs, the one or more computer programs may be performed and/or interpreted on a programmable system including at least one programmable processor. The programmable processor may be a dedicated or general programmable processor, which can receive data and instructions from a storage system, at least one input device, and at least one output device, and transmit the data and instructions to the storage system, the at least one input device, and the at least one output device.

Program codes used to implement the method of the present disclosure can be written in any combination of one or more programming languages. These program codes can be provided to the processors or controllers of general computers, special computers, or other programmable data processing devices, so that, when the program codes are performed by the processors or controllers, functions/operations specified in the flowcharts and/or block diagrams are implemented. The program codes can be performed entirely on a machine, partially performed on the machine, and partially performed on the machine and partially performed on a remote machine as an independent software package, or entirely performed on the remote machine or a server.

In the context of the present disclosure, a machine-readable medium may be a tangible medium, which may include or store a program for being used by an instruction execution system, device, or apparatus or in combination with the instruction execution system, apparatus, or device. The machine-readable medium may be a machine-readable signal medium or a machine-readable storage medium. The machine-readable medium may include, but is not limited to, an electronic, magnetic, optical, electromagnetic, infrared, or semiconductor system, device, or apparatus, or any foregoing suitable combinations. More specific examples of the machine-readable storage medium may include electrical connections based on one or more wires, a portable computer disk, a hard disk, a RAM, a ROM, an Erasable Programmable Read-Only Memory (EPROM or flash memory), an optical fiber, a portable Compact Disk Read-Only Memory (CD-ROM), an optical storage device, a magnetic storage device, or any above suitable combinations.

In order to provide interaction with a user, the system and technologies described herein can be implemented on a computer, including a display device for displaying information to the user (for example, a Cathode Ray Tube (CRT) or a Liquid Crystal Display (LCD) monitor), a keyboard and a pointing device (for example, a mouse or a trackball). The user can provide an input to the computer by using the keyboard and the pointing device. Other types of devices may also be configured to provide interaction with the user, for example, the feedback provided to the user may be any form of sensory feedback (such as visual feedback, auditory feedback, or tactile feedback), and may be the input from the user received in any form (including acoustic input, voice input, or tactile input).

The system and technologies described herein may be implemented in a computing system (for example, as a data server) including a back-end component, or a computing system (for example, an application server) including a middleware component, or a computing system (for example, a user computer with a graphical user interface or network browser, the user may be in interaction with implementations of the system and technologies described herein by using the graphical user interface or network browser) including a front-end component, or a computing system including any combination of the back-end component, the middleware component, or the front-end component. The components of the system can be connected with each other through any form or digital data communication (for example, a communication network) of the medium. Examples of the communication network include a Local Area Network (LAN), a Wide Area Network (WAN), and the Internet.

The computer system may include a client and a server. The client and the server are generally far away from each other and usually interact by means of the communication network. A relationship between the client and the server is generated by the computer program that is run on the corresponding computer and has a client-server relationship with each other.

It is to be understood that, the steps may be reordered, added or deleted by using various forms of programs shown above. For example, the steps described in the present disclosure may be performed parallelly, sequentially, or in a different order, as long as desired results of the technical solutions disclosed in the present disclosure can be achieved, which are not limited herein.

The foregoing specific implementations do not constitute limitations on the protection scope of the present disclosure. Those skilled in the art should understand that, various modifications, combinations, sub-combinations and substitutions can be made according to design requirements and other factors. Any modifications, equivalent replacements, improvements and the like made within the spirit and principle of the present disclosure shall fall within the scope of protection of the present disclosure.

What is claimed is:

1. An information processing method, comprising:
determining initial representation of edges connected between a plurality of atoms in a molecule based on three-dimensional structure information of the molecule;
determining first representation of a neighbor edge of each of the atoms based on the initial representation of the edges, the neighbor edge of each of the atoms indicating at least one edge connected with each of the atoms;
determining first representation of each of the atoms based on the first representation of the neighbor edge of each of the atoms; and
determining feature representation for characterizing the molecule based on the first representation of each of the atoms.

2. The method as claimed in claim 1, wherein determining the first representation of the neighbor edge of each of the atoms comprises:
determining an included angle between each of the other edges except a first edge in the neighbor edge of a first atom in the plurality of atoms and the first edge to obtain a plurality of included angles; and
determining the first representation of the first edge based on the plurality of included angles and initial representation of the other edges.

3. The method as claimed in claim 2, wherein determining the first representation of the first edge comprises:
dividing the other edges into different angle domains based on plurality of included angles;
determining weighted initial representation for each of the angle domains through weighted summation of the initial representation of the other edges in each of the angle domains based on attention weight of the other edges in each of the angle domains to the first edge; and
concatenating the weighted initial representation for each of the angle domains as the first representation of the first edge.

4. The method as claimed in claim 1, wherein determining the first representation of each of the atoms comprises:
determining a distance between the neighbor edge of the first atom in the plurality of atoms and the first atom, the distance between the neighbor edge and the first atom indicating a distance between a second atom connected with the neighbor edge and the first atom;
determining the attention weight of the neighbor edge of the first atom to the first atom based on the distance; and
determining an weighted average of the first representation of the neighbor edge of the first atom based on the attention weight as first representation of the first atom.

5. The method as claimed in claim 1, wherein determining the feature representation for characterizing the molecule comprises:
determining second representation of the edges based on the first representation of each of the atoms;
determining third representation of the neighbor edge of each of the atoms based on the second representation of the edges;
determining second representation of each of the atoms based on the third representation of the neighbor edge of each of the atoms; and
determining the feature representation for characterizing the molecule based on the second representation of each of the atoms.

6. The method as claimed in claim 1, wherein the three-dimensional structure information of the molecule is represented by a polar coordinate system.

7. The method as claimed in claim 1, wherein determining the initial representation of edges connected between the plurality of atoms in the molecule comprises:
determining initial representation of the plurality of atoms in the molecule based on the three-dimensional structure information of the molecule;
determining characterization of the distance between the plurality of atoms connected by the edge based on the three-dimensional structure information of the molecule; and
determining the initial representation of edges connected between the plurality of atoms based on the initial representation of the plurality of atoms and the characterization of the distance between plurality of the atoms.

8. The method as claimed in claim 1, further comprising:
determining whether a distance between any two atoms of the plurality of atoms is less than a threshold distance; and
in response to determining that the distance between these two atoms is less than the threshold distance, constructing an edge for connecting these two atoms.

9. The method as claimed in claim 1, wherein the three-dimensional structure information of the molecule at least comprises types and space distribution of the atoms forming the molecule.

10. The method as claimed in claim 1, wherein the initial representation of edges is one-dimensional vector, and the feature representation for characterizing the molecule is one-dimensional vector.

11. An electronic device, comprising:
at least one processor, and
a memorizer, in communication connection with the at least one processor, wherein
the memorizer stores an instruction capable of being performed by the at least one processor, and the instruction is performed by the at least one processor, to cause the at least one processor to perform the following steps:
determining initial representation of edges connected between a plurality of atoms in a molecule based on three-dimensional structure information of the molecule;
determining first representation of a neighbor edge of each of the atoms based on the initial representation of the edges, the neighbor edge of each of the atoms indicating at least one edge connected with each of the atoms;
determining first representation of each of the atoms based on the first representation of the neighbor edge of each of the atoms; and
determining feature representation for characterizing the molecule based on the first representation of each of the atoms.

12. The electronic device as claimed in claim 11, wherein determining the first representation of the neighbor edge of each of the atoms comprises:
determining an included angle between each of the other edges except a first edge in the neighbor edge of a first atom in the plurality of atoms and the first edge to obtain a plurality of included angles; and
determining the first representation of the first edge based on the plurality of included angles and initial representation of the other edges.

13. The electronic device as claimed in claim 12, wherein determining the first representation of the first edge comprises:
dividing the other edges into different angle domains based on plurality of included angles;
determining weighted initial representation for each of the angle domains through weighted summation of the initial representation of the other edges in each of the angle domains based on attention weight of the other edges in each of the angle domains to the first edge; and
concatenating the weighted initial representation for each of the angle domains as the first representation of the first edge.

14. The electronic device as claimed in claim 11, wherein determining the first representation of each of the atoms comprises:
determining a distance between the neighbor edge of the first atom in the plurality of atoms and the first atom, the distance between the neighbor edge and the first atom indicating a distance between a second atom connected with the neighbor edge and the first atom;
determining the attention weight of the neighbor edge of the first atom to the first atom based on the distance; and
determining an weighted average of the first representation of the neighbor edge of the first atom based on the attention weight as first representation of the first atom.

15. The electronic device as claimed in claim 11, wherein determining the feature representation for characterizing the molecule comprises:
determining second representation of the edges based on the first representation of each of the atoms;
determining third representation of the neighbor edge of each of the atoms based on the second representation of the edges;
determining second representation of each of the atoms based on the third representation of the neighbor edge of each of the atoms; and
determining the feature representation for characterizing the molecule based on the second representation of each of the atoms.

16. A non-transitory storage medium, storing a computer instruction, wherein the computer instruction is used for a computer to perform the following steps:
determining initial representation of edges connected between a plurality of atoms in a molecule based on three-dimensional structure information of the molecule;
determining first representation of a neighbor edge of each of the atoms based on the initial representation of the edges, the neighbor edge of each of the atoms indicating at least one edge connected with each of the atoms;
determining first representation of each of the atoms based on the first representation of the neighbor edge of each of the atoms; and
determining feature representation for characterizing the molecule based on the first representation of each of the atoms.

17. The non-transitory storage medium as claimed in claim 16, wherein determining the first representation of the neighbor edge of each of the atoms comprises:
determining an included angle between each of the other edges except a first edge in the neighbor edge of a first atom in the plurality of atoms and the first edge to obtain a plurality of included angles; and
determining the first representation of the first edge based on the plurality of included angles and initial representation of the other edges.

18. The non-transitory storage medium as claimed in claim 17, wherein determining the first representation of the first edge comprises:
dividing the other edges into different angle domains based on plurality of included angles;
determining weighted initial representation for each of the angle domains through weighted summation of the initial representation of the other edges in each of the angle domains based on attention weight of the other edges in each of the angle domains to the first edge; and
concatenating the weighted initial representation for each of the angle domains as the first representation of the first edge.

19. The non-transitory storage medium as claimed in claim 16, wherein determining the first representation of each of the atoms comprises:
determining a distance between the neighbor edge of the first atom in the plurality of atoms and the first atom, the distance between the neighbor edge and the first atom indicating a distance between a second atom connected with the neighbor edge and the first atom;
determining the attention weight of the neighbor edge of the first atom to the first atom based on the distance; and
determining an weighted average of the first representation of the neighbor edge of the first atom based on the attention weight as first representation of the first atom.

20. The non-transitory storage medium as claimed in claim 16, wherein determining the feature representation for characterizing the molecule comprises:
determining second representation of the edges based on the first representation of each of the atoms;
determining third representation of the neighbor edge of each of the atoms based on the second representation of the edges;
determining second representation of each of the atoms based on the third representation of the neighbor edge of each of the atoms; and
determining the feature representation for characterizing the molecule based on the second representation of each of the atoms.
